(19) United States
(12) Patent Application Publication
(10) Pub. No.: US 2024/0001031 A1
(43) Pub. Date: Jan. 4, 2024

(54) APPARATUSES AND METHODS FOR NORMALIZING LOADED PUMP MOTOR DATA TO UNLOADED PUMP MOTOR DATA DURING A FLUID MOVEMENT OPERATION

(71) Applicant: Becton, Dickinson and Company, Franklin Lakes, NJ (US)

(72) Inventors: Scott STEWART, Pittsburgh, PA (US); Scott COREY, Hydes, MD (US); Kun LI, Boyds, MD (US)

(73) Assignee: Becton, Dickinson and Company, Franklin Lakes, NJ (US)

(21) Appl. No.: 18/255,293

(22) PCT Filed: Dec. 9, 2021
(86) PCT No.: PCT/US2021/062562

§ 371 Date: May 31, 2023

Related U.S. Application Data
(60) Provisional application No. 63/125,508, filed on Dec. 15, 2020.

(51) Int. Cl. A61M 5/168; A61M 5/142

(52) U.S. Cl. CPC A61M 5/16854; A61M 5/14216; A61M 5/16809; A61M 2005/16863

(57) ABSTRACT

Devices and methods are provided to improve accuracy of occlusion detection based on measured data in a fluid delivery device by using dead band normalization of loaded measured data to unloaded measured data. The loaded measured data and unloaded measured data are obtained during the same fluid movement operation or stroke of the fluid delivery device. The dead band normalization can be performed during an aspiration operation or a dispensing operation. An interface in the fluid delivery device that is close to the fluid driving mechanism and that can at least temporarily move under control but without moving fluid can be used to identify when to generate unloaded measured data during a fluid movement operation for dead band normalization of loaded measured data measured during that fluid movement operation.

### BACKGROUND

### Field

The present disclosure and technical solution described herein generally relate to performing dead band normalization by normalizing loaded measured data related to fluid movement (e.g., pump motor current measured during a fluid dispense or aspirate operation when fluid drive mechanism components are controlled to move fluid) to unloaded measured data obtained when the drive mechanism components are not moving fluid during that dispense or aspirate operation, and to detecting occlusion using dead band normalization.

### Description of Related Art

Current sensing is a method of detecting occlusions in the fluid path of a fluid delivery device such as an infusion pump because an occlusion causes a decrease in flow which causes increased pressure. Increased pressure causes increased torque demand on the pump motor, and increased torque demand by the motor draws more current. Other motor parameters besides motor current such as motor voltage and encoded count can be used to detect increased pressure.

However, many other design factors affect current demand by motors, as well as other motor parameters, including, but not limited to, gearbox efficiency, pump seals and their wear over time, motor efficiency, and motor magnet angle. In addition, there are environmental factors like ambient pressure and temperature that can affect motor current demand. These factors can negatively impact the accuracy of using a measured pump motor parameter such as motor current to detect occlusion.

### SUMMARY

The above and other problems are overcome, and additional advantages are realized, by illustrative embodiments.

In accordance with aspects of illustrative embodiments, a fluid delivery device is provided that comprises: a pump comprising a chamber of fluid, and a drive mechanism configured to control movement of a designated volume of fluid with respect to the chamber during a fluid movement operation; and a processing device configured, during a fluid movement operation, to generate measured data comprising unloaded measured data obtained during a portion of the fluid movement operation wherein the pump does not move fluid, and loaded measured data obtained while the pump is moving fluid during the fluid movement operation, the measured data being indicative of fluid movement in the pump, and to normalize the loaded measured data to the unloaded measured data.

In accordance with aspects of illustrative embodiments, the processing device is further configured to analyze the normalized loaded measured data to determine if it satisfies a designated metric related to pressure in the infusion device that indicates occlusion.

In accordance with aspects of illustrative embodiments, the processing device is further configured, during a subsequent fluid movement operation by the pump to generate unloaded measured data during a portion of the subsequent fluid movement operation wherein the pump does not move fluid, generate loaded measured data while the pump is moving fluid during the subsequent fluid movement operation, the measured data being indicative of fluid movement in the pump, and normalize the loaded measured data to the unloaded measured data.

In accordance with aspects of illustrative embodiments, the fluid movement operation is an incremental operation among a plurality of fluid movement operations to dispense fluid from the chamber or aspirate fluid into the chamber.

In accordance with aspects of illustrative embodiments, the processing device is further configured to normalize the loaded measured data to the unloaded measured data for each fluid movement operation of the fluid delivery device, or least for a selected subset of fluid movement operations of the fluid delivery device.

In accordance with aspects of illustrative embodiments, the fluid delivery operation is chosen from an aspirate operation to draw fluid into the chamber and a dispense operation to expel fluid from the chamber.

In accordance with aspects of illustrative embodiments, the measured data indicates a fluid characteristic chosen from fluid pressure and fluid flow rate.

In accordance with aspects of illustrative embodiments, the pump is a syringe-type pump having a barrel as the chamber and a plunger and the drive mechanism is operable to selectively drive the plunger to dispense fluid from the barrel, and the processing device is configured to generate the unloaded measured data before the measured data indicates that fluid pressure or flow rate has begun to increase from driving the plunger by the drive mechanism during the fluid movement operation.

In accordance with aspects of illustrative embodiments, the pump is characterized by an interface comprising at least one or more components in the drive mechanism and the operation of which causes the portion within a fluid movement operation wherein the pump does not move fluid to occur.

In accordance with aspects of illustrative embodiments, the pump can be a syringe-type pump having a barrel as the chamber and the interface comprises a plunger, the drive mechanism being operable to selectively drive the plunger to dispense fluid from the barrel, and the processing device is configured to generate the unloaded measured data during a dispensing fluid movement operation by temporarily retracting the plunger in the barrel a nominal amount.

In accordance with aspects of illustrative embodiments, the pump can be a syringe-type pump having a barrel as the chamber and the interface comprises a plunger, the drive mechanism being operable to selectively drive the plunger to dispense fluid from the barrel, and the processing device is configured to generate the unloaded measured data during an aspirating fluid movement operation by manual or externally controlled filling of the barrel via an inlet port to the barrel, and to generate the loaded measured data during the aspirating fluid movement operation by controlling the pump to temporarily retract the plunger within the barrel.

In accordance with aspects of illustrative embodiments, the pump can be a syringe-type pump having a barrel as the chamber and a plunger, the interface comprises a pusher coupled to the drive mechanism, the drive mechanism being operable to selectively drive the pusher to abut the plunger to dispense fluid from the barrel, and the processing device is configured to generate the unloaded measured data during a dispensing fluid movement operation by temporarily retracting the pusher in the barrel.

In accordance with aspects of illustrative embodiments, the pump can be a syringe-type pump having a barrel as the chamber and a plunger, the interface comprises a pusher coupled to the drive mechanism, the drive mechanism being operable to selectively drive the pusher to abut the plunger to dispense fluid from the barrel, and the processing device is configured to generate the unloaded measured data prior to gathering loaded measured data by incrementing through a known number of dispense cycles in which the pusher has not yet hit the plunger

In accordance with aspects of illustrative embodiments, the pump can be a rotational metering-type pump comprising an inlet port and an outlet port and wherein the drive mechanism is connected to a pump motor via a gearbox and the chamber has at least one aperture, the drive mechanism being operable to selectively drive a piston to dispense fluid from or aspirate fluid into the chamber and to control cooperation of the at least one aperture with the inlet port during an aspirating fluid movement operation and with the outlet port during a dispensing fluid movement operation, the interface comprising a feature on the drive mechanism that is configured to cooperate with the gearbox to enable the drive mechanism to not move fluid with respect to the chamber during at least a portion of the aspirating fluid movement operation and the dispensing fluid movement operation.

In accordance with aspects of illustrative embodiments, the pump can be a rotational metering-type pump and the interface comprises a pin on a piston that is controllably inserted and retracted within a sleeve and a helical groove in the sleeve, the drive mechanism being operable to rotate the sleeve causing the for controlling fluid volume in the chamber via a helical groove in the sleeve to guide the pin to translate along the helical groove to guide the retraction and insertion of the piston within the sleeve to control fluid volume of the chamber, the pin and/or groove being configured to enable the piston to not move fluid with respect to the chamber during at least a portion of a fluid movement operation.

In accordance with aspects of illustrative embodiments, the interface comprises a cam coupled to the drive mechanism, and the processing device is configured to generate the unloaded measured data during a fluid movement operation when a cam follower connected to an actuator for the drive mechanism traverses at least part of a flat portion of the cam resulting in no fluid movement during the fluid movement operation.

In accordance with aspects of illustrative embodiments, the pump has a reservoir as the chamber, a plunger and a drive mechanism operable to selectively drive the plunger to dispense fluid from the reservoir, and the processing device is configured with baseline data related to a designated waveform of the measured data during fluid movement operations, the waveform having a dead portion therein corresponding to when fluid pressure or rate from driving the plunger by the drive mechanism has not yet begun to increase, the processing device being configured to analyze the measured data using the baseline data to determine when to generate the unloaded measured data during a fluid dispense operation.

Additional and/or other aspects and advantages of illustrative embodiments will be set forth in the description that follows, or will be apparent from the description, or may be learned by practice of the illustrative embodiments. The illustrative embodiments may comprise apparatuses and methods for operating same having one or more of the above aspects, and/or one or more of the features and combinations thereof. The illustrative embodiments may comprise one or more of the features and/or combinations of the above aspects as recited, for example, in the attached claims

### BRIEF DESCRIPTION OF THE DRAWINGS

The above and/or other aspects and advantages of the illustrative embodiments will be more readily appreciated from the following detailed description, taken in conjunction with the accompanying drawings, of which:

FIGS. 1A and 1B depict, respectively, raw and filtered data (e.g., motor current) from an example fluid delivery device during aspirate and dispense strokes;

FIG. 1C depicts filtered measured data (e.g., motor current) from an example fluid delivery device during dispensing and variance at different pressures;

FIG. 1D depicts measured data (e.g., motor current) during operation of an example fluid delivery device with its drive mechanism component(s) moving fluid and not moving fluid to illustrate a dead band normalization region in the data

FIG. 2 depicts measured data (e.g., motor current) from an example fluid delivery device during a dispense operation and data from region therein identified for dead band normalization.

FIG. 3 is a flow chart of illustrative operations of an example fluid delivery device performing a dispense operation with dead band normalization of measured data in accordance with an illustrative embodiment.

FIG. 4 is a perspective view of an example wearable fluid delivery device employing an occlusion detection algorithm with dead band normalization in accordance with an example embodiment;

FIGS. 5A, 5B, 5C and 5D are, respectively, a partial top view, a perspective view, a side view, and a top view of the example fluid delivery device of FIG. 1 with the cover removed;

FIG. 6 is a block diagram of example components of an example fluid delivery device constructed in accordance with an example embodiment;

FIGS. 7A, 7B, 7C and 7D are perspective top views of an example fluid delivery device with the cover removed and showing different stages of filling a reservoir;

FIGS. 8A and 8B are, respectively, a front perspective view and a rear perspective view of a plunger driver component constructed in accordance with an example embodiment;

FIG. 9 is a side view of the plunger driver assembly of the example fluid delivery device of FIGS. 7A-7D shown in a retracted position;

FIGS. 10A, 10B, 10C and 10D are perspective top views of the example fluid delivery device of FIGS. 7A-7D with the cover removed and showing different stages of discharging fluid from a reservoir via the plunger drive assembly;

FIG. 11 is a perspective view of a center screw with keying feature constructed in accordance with an example embodiment for cooperating with the plunger driver component in FIGS. 8A-8B;

FIGS. 12 and 13 are partial, perspective views of example pump components in an example fluid delivery device that operates in accordance with an occlusion detection algorithm using dead band normalization in accordance with an illustrative embodiment;

FIGS. 14A and 14B are perspective views of pump components of FIGS. 12 and 13 in an example fluid delivery device arranged, respectively, in accordance with a ready to dispense stage of operation and a ready to aspirate stage of operation;

FIG. 14C is a perspective view of components in an example fluid delivery device comprising example pump components of FIGS. 12 and 13 and associated electronic circuits on a printed circuit board;

FIG. 14D is a partial perspective view of an example motor and gearbox assembly configured to cooperation with the pump components of FIGS. 12 and 13;

FIG. 15A is a block diagram of components in an example fluid delivery device; and

FIG. 15B is a schematic diagram of a fluid delivery device pump motor having a current sensor in accordance with an illustrative embodiment.

Throughout the drawing figures, like reference numbers will be understood to refer to like elements, features and structures.

### DETAILED DESCRIPTION OF ILLUSTRATIVE EMBODIMENTS

As will be appreciated by one skilled in the art, there are numerous ways of carrying out the examples, improvements, and arrangements of a fluid delivery device in accordance with embodiments disclosed herein. Although reference will be made to the illustrative embodiments depicted in the drawings and the following descriptions, the embodiments disclosed herein are not meant to be exhaustive of the various alternative designs and embodiments that are encompassed by the disclosed technical solutions, and those skilled in the art will readily appreciate that various modifications may be made, and various combinations can be made with departing from the scope of the disclosed technical solutions.

Example embodiments in the present disclosure provide a technical solution to the above described problems. Because of the afore-mentioned design and environmental factors that impact demand on a pump motor, it is extremely important to adjust or calibrate a pump motor signal used to detect occlusions in a fluid delivery device such as an infusion pump so that only changes to the motor signal that are due to changes in pressure are measured and used for occlusion detection and not factors unrelated to pressure such as changes over time in the battery, motor and gearbox from wear, ambient temperature changes, differences in pump performance during aspirate versus dispense operations, and so on. An ideal normalization compensates for everything but pressure, and the example embodiments and technical solution provided herein are advantageously close to ideal normalization.

The technical solution and example embodiments provided in the present disclosure employ dead band normalization; that is, adjusting or normalizing measured data related to a fluid movement operation controlled by a drive mechanism in a fluid delivery device to data obtained during a dead portion of that operation when the drive mechanism is not moving fluid). The technical solution and example embodiments provided in the present disclosure advantageously employ dead band normalization to improve accuracy of detecting occlusion or other condition by using the normalized measured data. The measured data can be, for example, motor current during a fluid dispense or aspirate operation. As used herein, “loaded” measured data refers to the measured data obtained during a fluid movement operation when the drive mechanism is moving fluid, and “unloaded” measured data refers to the measured data obtained during a dead portion of the fluid movement operation when fluid is not being moved by the drive mechanism. Dead band normalization is understood to mean that loaded measured data is adjusted or normalized to the unloaded measured data during a particular fluid movement operation in a fluid delivery device. “Dead band normalizing” and “dead band normalization” as used herein are advantageous because they remove unwanted signal noise components and/or the effects of undesirable variants related to the drive mechanism in a fluid delivery device (e.g., a pump motor in a medication infusion device) from measured data. Removal of unwanted signal noise or undesirable impacts of noise factors (e.g., motor design or environmental factors) from measured r data can involve, for example, subtracting a averaged unloaded measured data from loaded measured data obtained while a pump motor is operated to move fluid. Dead band normalization can also involve other mathematical adjustment or calibration operations besides subtraction to normalize measured loaded pump motor data to measured unloaded pump motor data such as dividing an averaged loaded measured signal by an averaged unloaded measured signal.

While there may be different options for normalizing measured datar such as pump motor current in a fluid delivery device, dead band normalizing to a dead band region of the measured data, as illustrated by example embodiments described below, realizes significant advantages in terms of accuracy of detecting a selected delivery device condition based on measured data. For example, one way to normalize pump motor data might be to normalize the measured data obtained during a dispense operation of the pump to the data obtained during a previous aspirate operation because the aspirate operation is not affected by downstream pressure. However, the aspirate operation is affected by different factors than a dispense operation such as upstream pressure, reservoir fill volume, and other noise factors which do not affect aspirate and dispense operations evenly. For example, normalizing measured pump motor data to an aspirate operation of the pump effectively doubles the noise in the measured signal and introduces noise factors not present with dead band normalization as provided by the technical solution described in the present disclosure.

Different factors impacting motor current are, for example, motor winding resistance, applied voltage (e.g., which changes with battery age), and motor speed. In addition, current during a dispense or aspirate operation can be impacted by gear train losses, motor friction losses, and drive mechanism (e.g., piston) friction losses. An advantage of the technical solution described herein is that a desired factor (i.e., pressure during an aspirate operation P_{A} or a dispense operation P_{D}) can be obtained by removing all of the other constants and factors related to friction losses and battery changes using dead band normalization in accordance with technical solution and example embodiments thereof described herein.

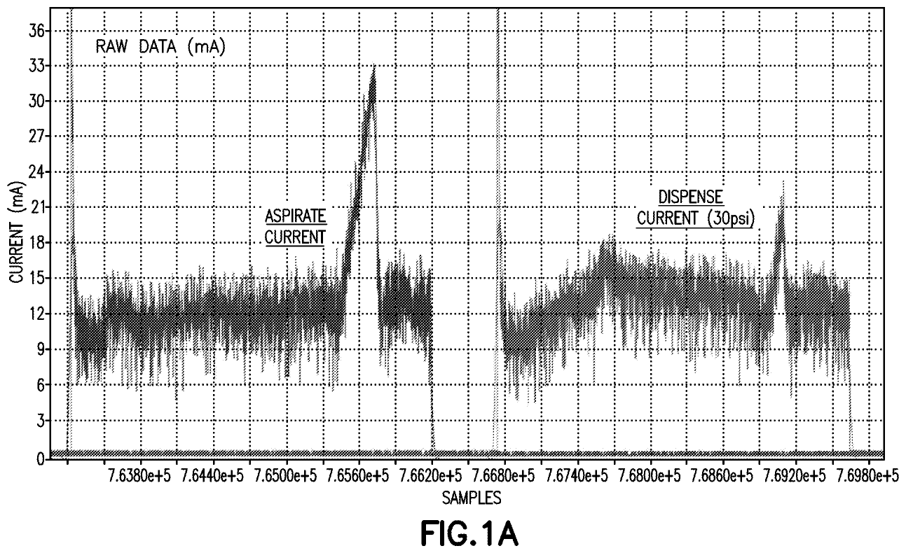

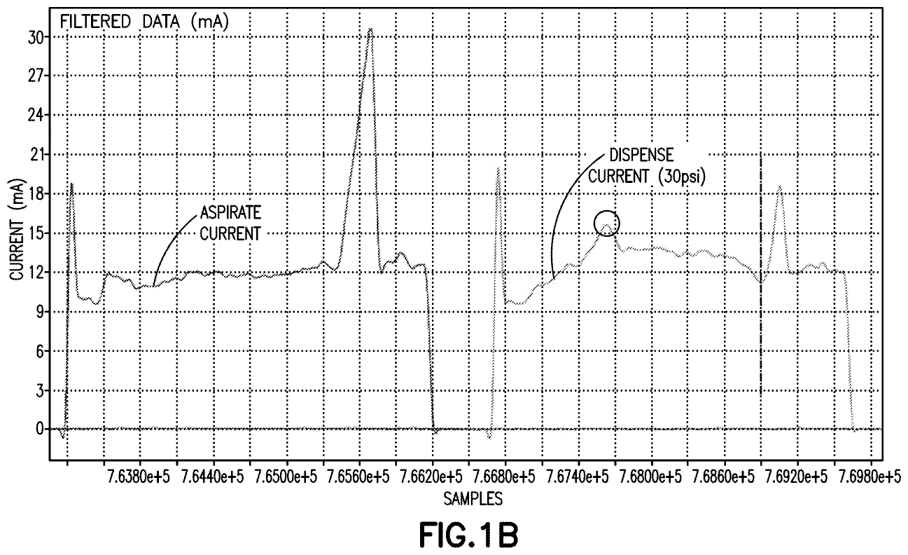

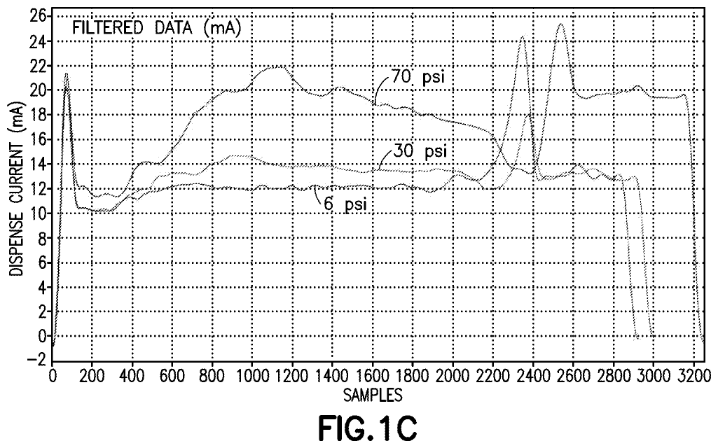

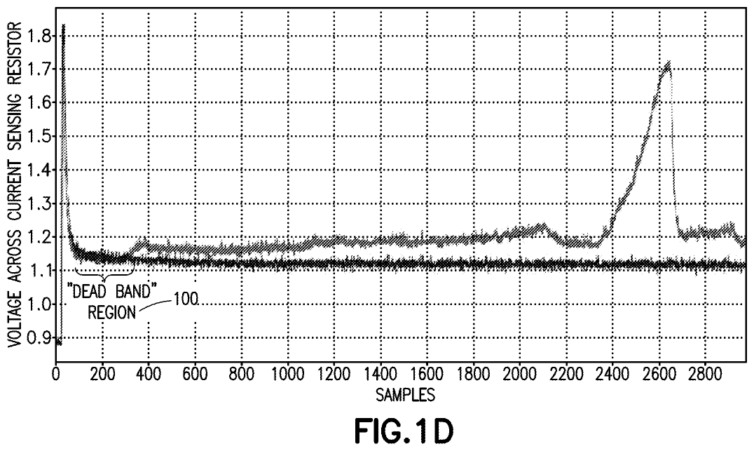

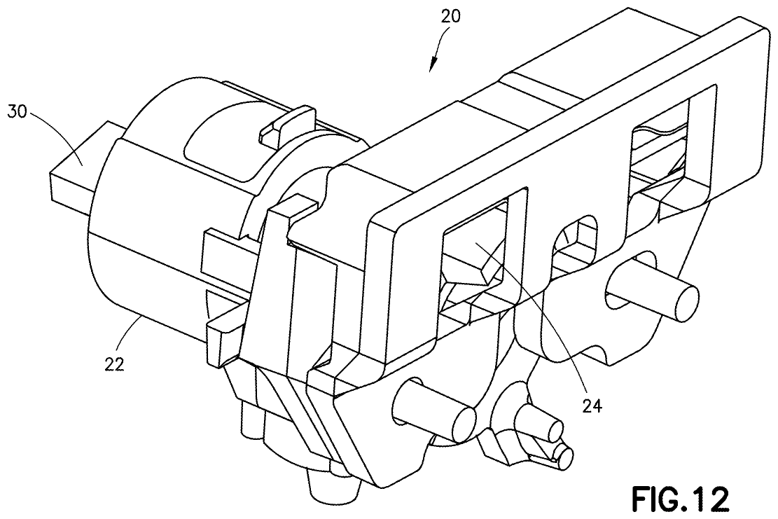

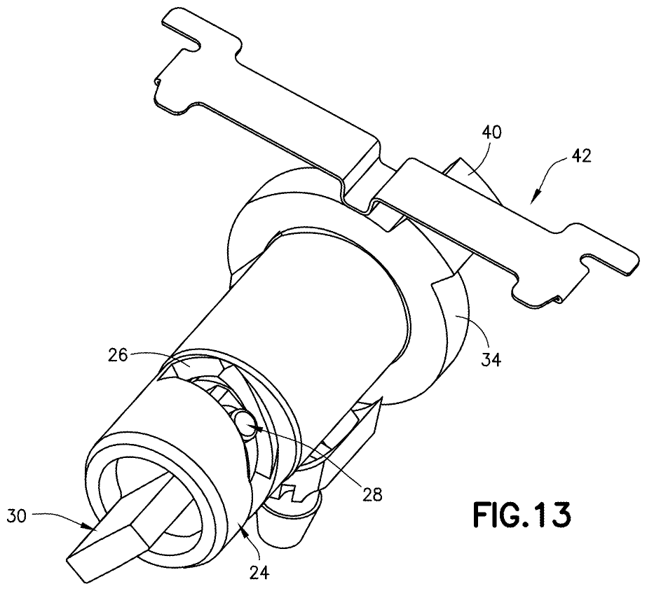

See, for example, FIGS. 1A and 1B that depict, respectively, raw and filtered pump measurement data (e.g., motor current) from an example fluid delivery device during aspirate and dispense operations. FIG. 1C depicts filtered pump measurement data from an example fluid delivery device indicating motor current during dispensing and variance at different pressures; however, all of the depicted measured current signals share a relatively similar waveform shape comprising a first spike 102 corresponding to motor start up, a portion 104 of the shape corresponding to piston movement, followed by a portion 106 corresponding to a valve state change and an interlock torque spike 108 related to a rotational metering-type pump described below in connection with FIGS. 12-15B.

Dead band normalization of a measured pump motor signal such as a motor current signal during a dispense operation involves obtaining the current signal when the motor gearbox is turning but is not engaging the pump. See, for example, FIG. 1D wherein two superimposed current signal waveforms are shown. One of the waveforms 112 is obtained during a dispense operation of the rotational metering-type pump, for example, and comprises a motor start-up spike 102, a piston movement portion 104, a valve state change 106 and interlock torque spike 108). The other waveform 110 is obtained during motor operation without the pump (e.g., the motor is disengaged from the pump drive mechanism). Both of these waveforms have a similar portion 100 corresponding to a dead band region that can be identified, and the data obtained therein during a dispense operation can be used for dead band normalization of measured pump motor data obtained during motor and pump operations to more accurately detect occlusion conditions by reducing noise with removal of undesirable variability of factors impacting the motor. Dead band normalizing to this part of the signal is good because: (1) this part of the measured pump motor parameter signal is nearer in time to the portion of the measured signal that is of interest (e.g., measure current during piston movement to determine pressure changes that may indicate occlusion) which inherently reduces noise because noise factors change with time; and (2) variations in the battery, motor, and gearbox are dead band normalized out of the analyzed signal because it is essentially the same as the signal from just those components. A noise factor(s) is understood to mean a factor(s) that introduces variability either internally or externally to the fluid delivery device system, or subsystem, or part thereof such as temperature, humidity, part-to-part variation, part wear, etc.

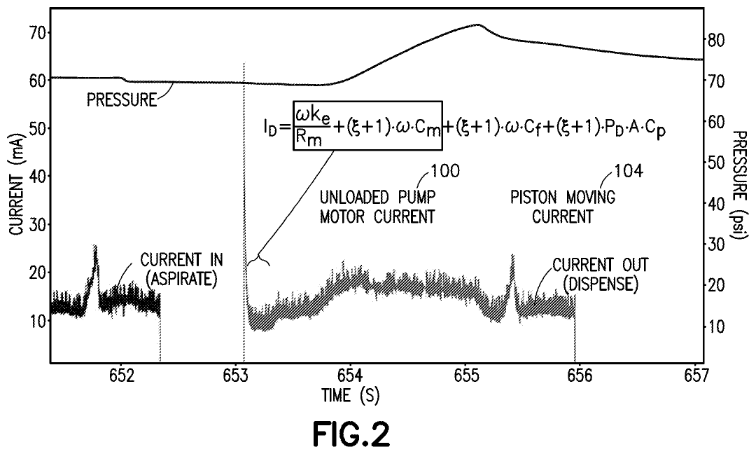

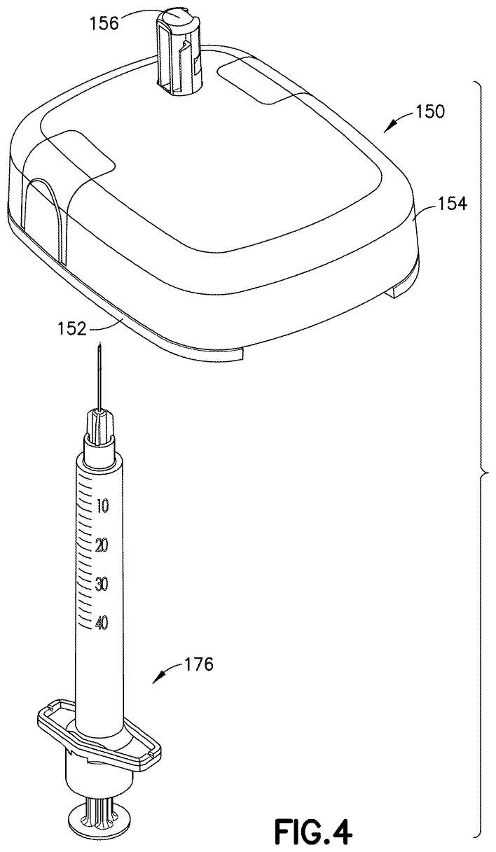

It is to be understood that the dead band region 100, or timing during a dispense or aspirate operation for obtaining dead band normalization data, can differ depending on the type of pump and pump drive mechanism. For example, a syringe-type pump as explained below in connection with FIGS. 4-11 can be operated, at any time during a dispense operation, to temporarily disengage the pump drive mechanism (e.g., reverse its direction so that it is not pushing a plunger in a syringe-type reservoir to dispense the fluid) to obtain unloaded measured data while fluid is not being moved during the dispense operation. This unloaded measured data, in turn, is used to dead band normalize loaded measured pump motor data obtained during pump engagement that results in dispensing of fluid. For a syringe-type pump that is filled manually (e.g., by a supply syringe coupled to an inlet port of the syringe-type reservoir of the pump), the motor can be controlled to perform a controlled aspirate operation wherein a controlled retraction of the pump piston draws back a plunger in the syringe-type reservoir to controllably intake more fluid from the supply reservoir into the fluid chamber of the syringe-type reservoir to obtain loaded measured data while fluid is being moved. This loaded measured data during a controlled aspirate movement can be dead band normalized to unloaded measured data obtained during manual filling when the drive mechanism is not being operated to move fluid. Alternatively, for a rotational metering-type pump as explained below in connection with FIGS. 12-15B, the dead band region 100 can occur at the beginning of each aspirate stroke and each dispense stroke as described above in connection with FIGS. 1B, 1D and 2.

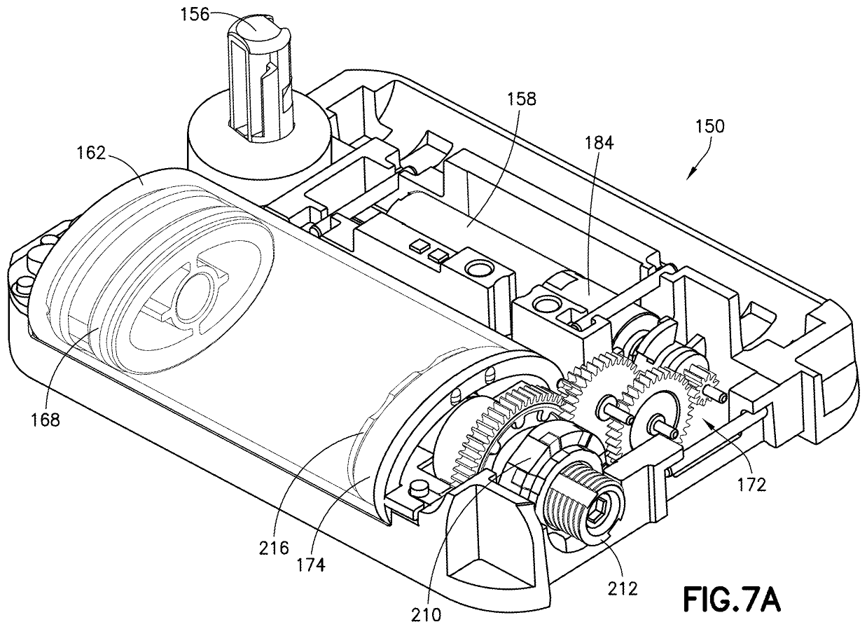

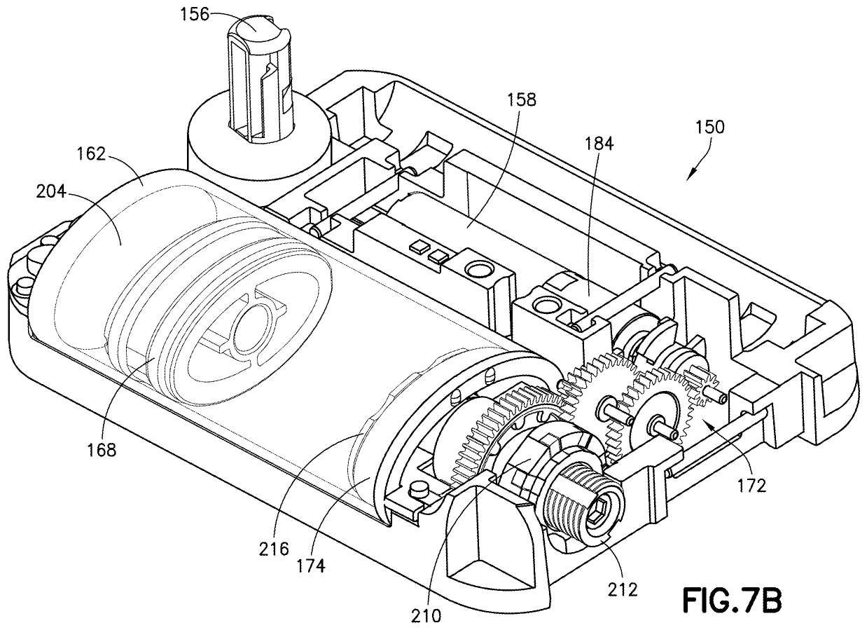

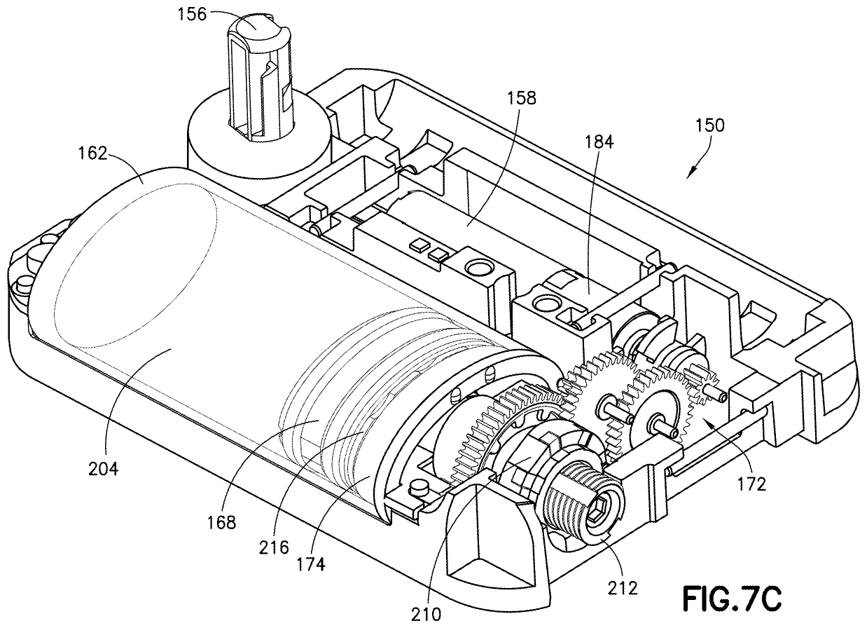

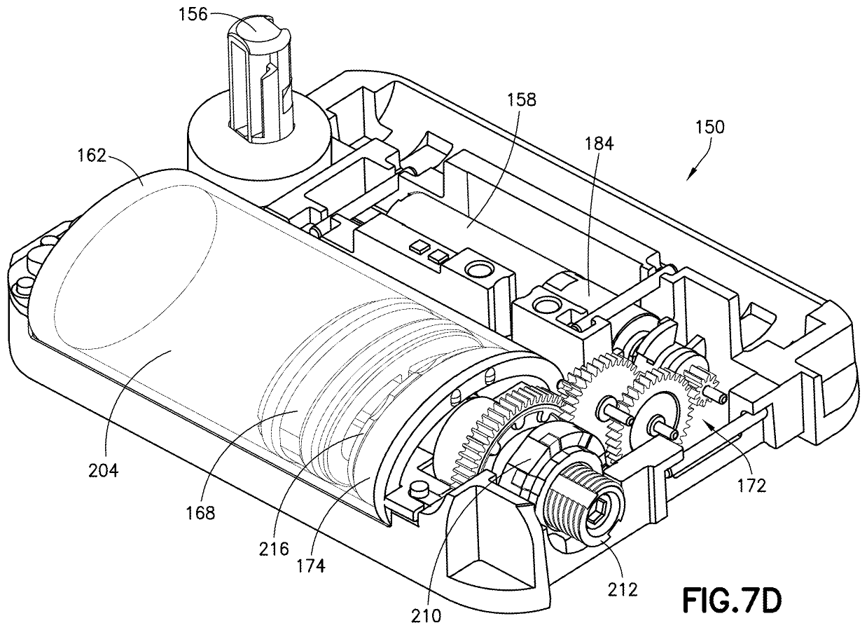

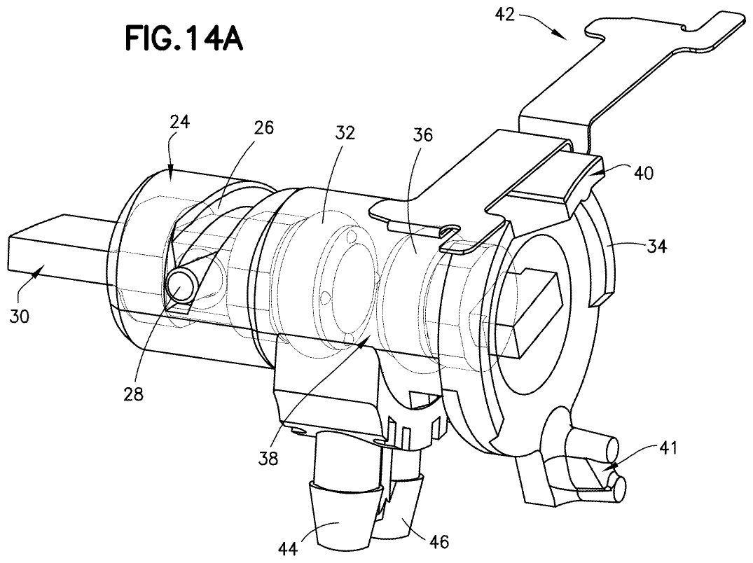

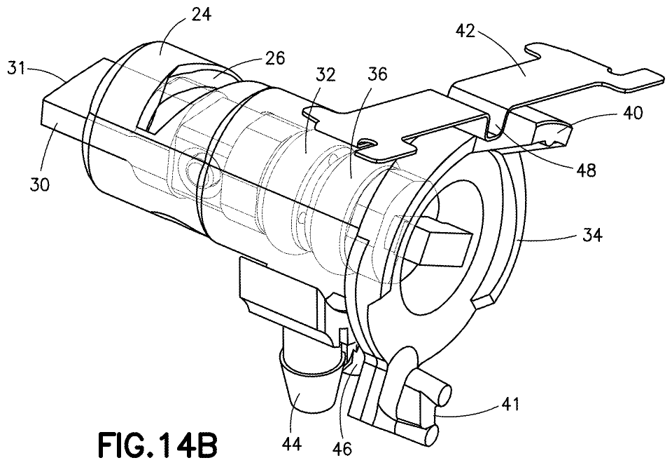

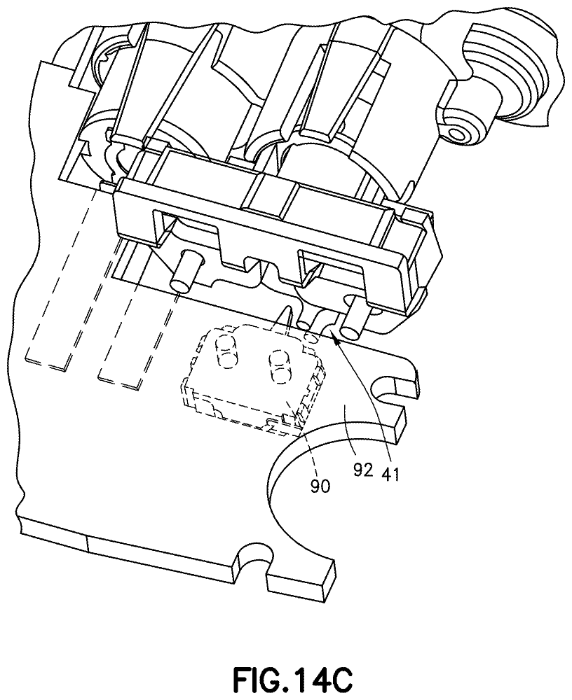

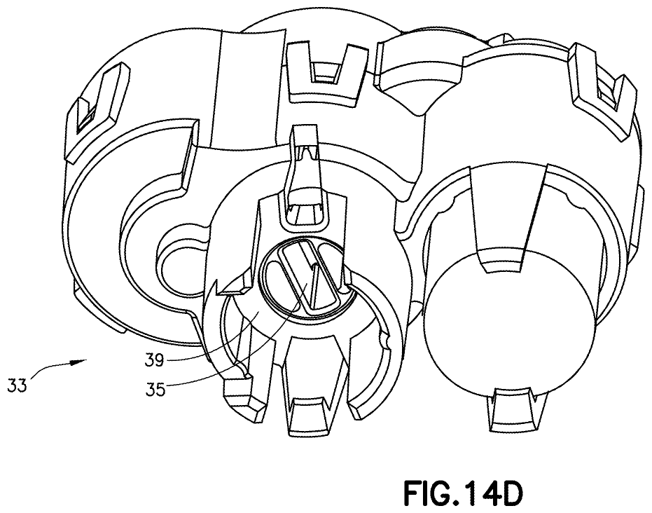

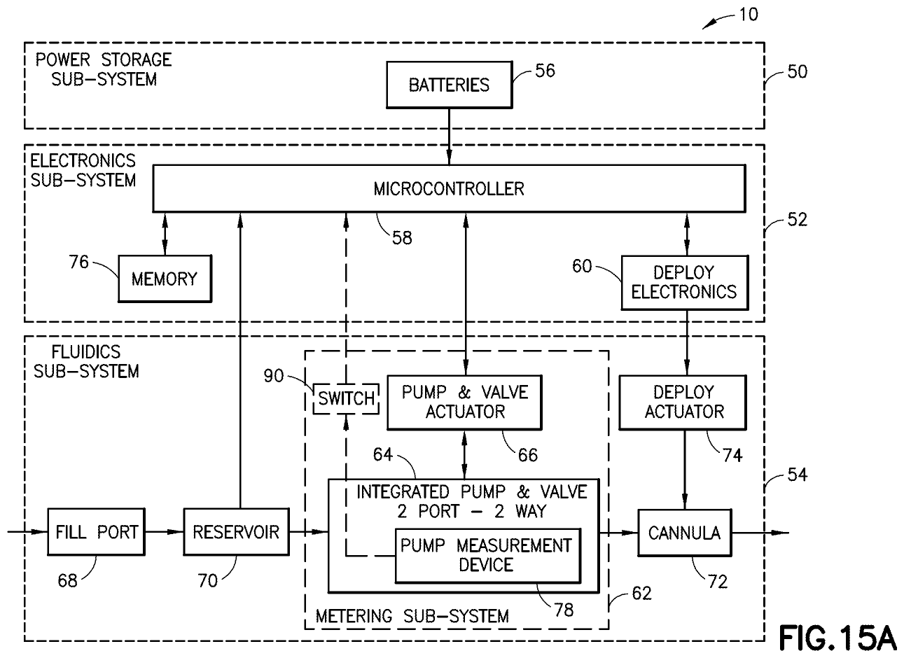

To optimize use of dead band normalization in accordance with the technical solution provided herein, the fluid delivery device has an interface as close to its fluid driving interface as possible that can move without moving the fluid. As described below in connection with FIGS. 7A-7B in the case of a syringe-type pump, this interface can be a plunger driver component such as a pusher 216 configured on the end of a drive mechanism that can abut a reservoir plunger 168 after the reservoir 162 is filled and be controlled to push the plunger 168 towards the distal end of the reservoir to dispense fluid therefrom. For dead band normalization, the pusher 216 can be driven backward and thereby disengaged from the plunger 168. During a dispense operation, the pusher 216 can be driven forward again to reengage the plunger 168. By being driven backward, the controller operating in accordance with a dead band normalization algorithm has essentially of all of the same effects of the pump motor driving the pusher 216 forward (e.g., to dispense) except for plunger 168 friction and force that results from pressure. In the case of a syringe-type pump configured without a pusher 216 (e.g., its drive mechanism is connected directly to its plunger), the interface can be the plunger being retracted during dispensing by a nominal amount to obtain unloaded measured data without causing unwanted retrograde fluid flow. As described below in connection with FIG. 14D in the case of a rotational metering-type pump, this interface can be a gap between an output gear of the gearbox and a piston tab such that, when the gearbox changes direction, there is a period of time when the piston is not engaged at all (e.g., dead band region 100 in FIG. 2) and therefore facilitates dead band normalization to work. Thus, the technical solution and example embodiments thereof described in the present disclosure advantageously employs a loose fitting drive train feature to obtain and use unloaded data from a dead band region of a fluid movement operation and therefore is very different from existing methods of reducing noise and efforts to improve accuracy of pump motor data readings.

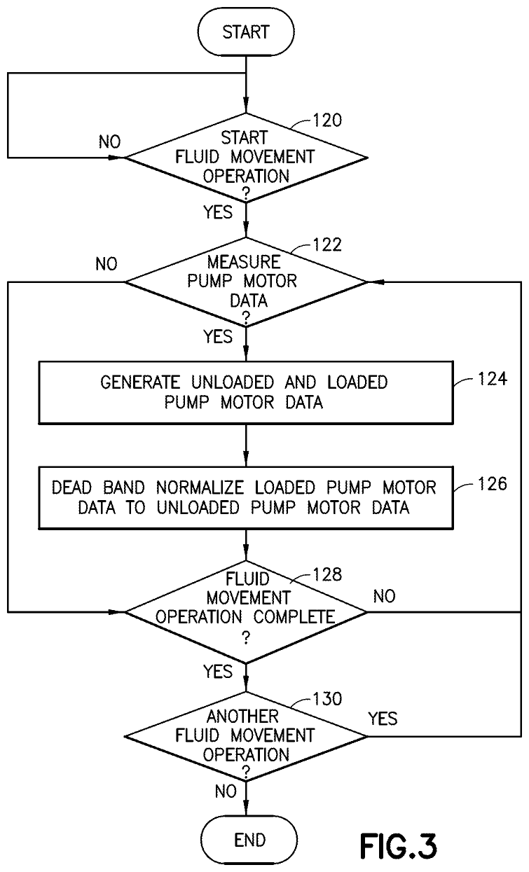

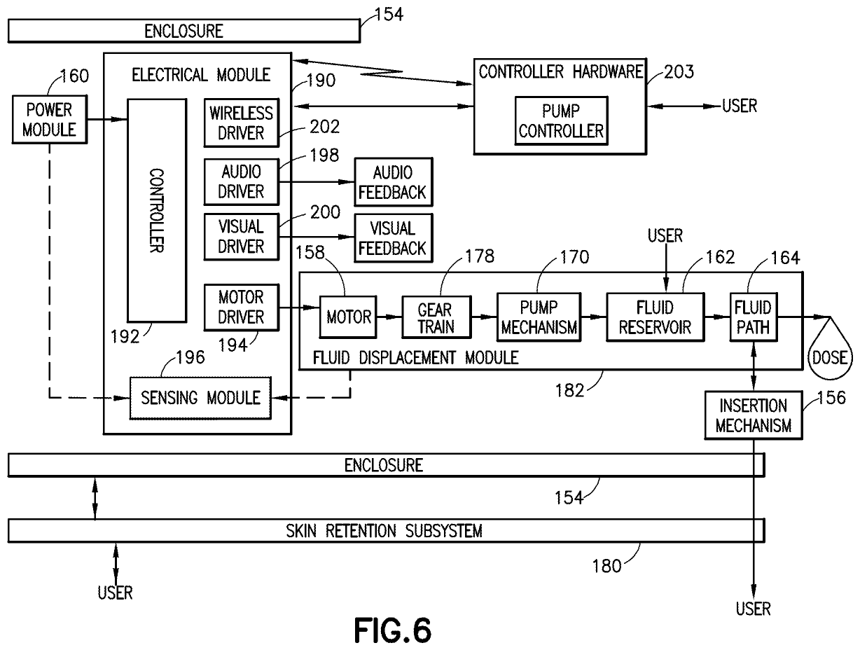

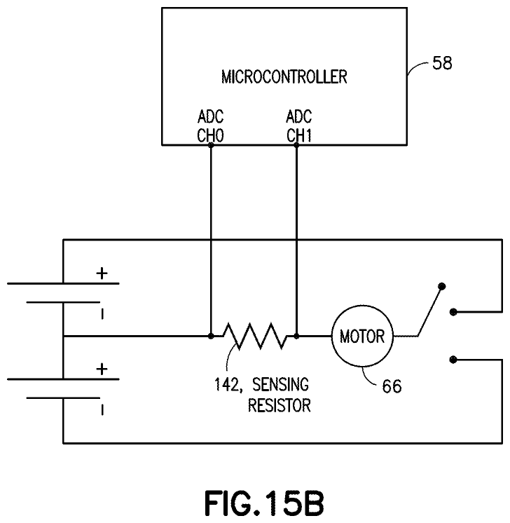

Operations associated with dead band normalization in accordance with illustrative embodiments of the technical solution described herein are shown in FIG. 3 and can be implemented, for example, as a dead band normalization algorithm performed by a controller (e.g., controller 192 in FIG. 6, or microcontroller 58 in FIG. 15A) or other device processing measured data. In accordance with an example embodiment, a controller for the fluid delivery device can be programmed or otherwise configured to obtain “loaded” measured data when drive mechanism is being operated for fluid movement (e.g., controlled intake or aspirating, or controlled output or dispensing), and normalize it with dead band or “unloaded” measured data obtained when the drive mechanism is being controlled for a fluid movement operation but momentarily is not moving fluid. In accordance with an example embodiment, dead band normalization is performed during an aspirate operation, a dispense operation, or during both types of operations. It can be beneficial to do it during fill and during delivery (e.g., disengage and reengage during any part of the overall aspirate or dispense stroke, depending on controlled volume intended to be drawn into the fluid chamber or delivered from fluid chamber). In any event, the dead band normalization data (e.g., the unloaded measured data) and the loaded measured data are optimally obtained during the same pump aspirate or dispense operation or stroke.

As illustrated in block 120 of FIG. 3, a pump controller can be configured, at the beginning of a fluid movement operation (e.g., an aspirate operation or stroke, or a dispense operation or stroke) to measure pump motor data related to that fluid movement operation (block 122). In accordance with an advantageous aspect of example embodiments of a technical solution described herein, the controller obtains or otherwise generates pump motor data comprising unloaded measured data and loaded measured data during that fluid movement operation (block 124). The controller performs dead band normalization in accordance with the technical solution described herein by normalizing the loaded measured data to the unloaded measured data corresponding to that fluid movement operation (block 126). It is to be understood that the dead band normalization can involve, for example, subtracting unloaded measured data from measured pump motor data to be able to advantageously determine fluid pressure or flow rate of the pump during that fluid delivery operation without being impacted by signal noise or noise factors (e.g., pump design and environmental factors). The unloaded measured data can be obtained at any point during the fluid movement operation wherein the drive mechanism does not move fluid. The loaded measured data can be obtained at multiple points during the fluid movement operation wherein the drive mechanism is engaged in moving fluid. In any event, the loaded and unloaded measured data obtained for that the fluid movement operation need not be used or relevant to a different fluid movement operation (block 130).

As stated before, the technical solution described herein successfully compensates for many changes in a pump (e.g., design factors of the battery, motor, and gearbox, and environment factors such as temperature) that are not related to changes in fluid pressure or flow rate or other measured parameter being used to detect occlusion or other condition of the fluid delivery device. An ideal normalization compensates for everything but pressure or flow rate, and this technical solution achieves essentially ideal normalization via dead band normalization illustrated in accordance with the example embodiments herein. As explained above with respect to the factors impacting motor current, for example, there are many terms and forces that ultimately add up to what current is measured, and the more of these terms or forces that can be normalized, the more accurate that occlusion detection using a measured parameter can be. Further, dead band normalizing as described herein allows an occlusion detection algorithm employing dead band normalization to evaluate individual fluid movement strokes or operations of a pump without having to look at changes over multiple strokes. Currently, there is no covering occlusion detection in infusion pumps related to current sensing or other measured pump motor parameter that utilizes a non-driving portion of the fluid movement to better assess fluid pressure or flow rate based on current or other measured motor parameter.

Occlusion in a fluid delivery device such as an infusion pump for medication can result from restricted flow or pathway constriction such as a pinched catheter or tissue occlusion, or from an empty medication reservoir. It is important to measure fluid pressure or flow rate changes in the fluid delivery device from an occlusion or other pump malfunction for early detection to mitigate against possible fluid delivery inaccuracies resulting therefrom such as missed doses. The technical solution and example embodiments herein achieve more accurate and faster detection of occlusion and therefore fewer fluid delivery inaccuracies.

The measured data is indicative of pressure or flow rate and can be, but is not limited to, any of motor current, motor voltage, encoder count, motor drive count, delivery pulse energy, motor drive time, and so on. For example, current sensing is generally considered to be a reliable method of detecting occlusions in a fluid path of a fluid delivery because motor current can be indirectly correlated to pressure. As stated above, an occlusion causes a decrease in fluid flow in the fluid delivery device, which causes increased pressure. An increase in pressure causes an increase in torque demand required by the motor to overcome this pressure. The increase in torque demand corresponds to an increase in current drawn by the motor, which is one way to detect occlusions such as an occluded catheter, or air in the fluid path, or malfunction of the motor.

FIGS. 4-11 illustrate an example fluid delivery device having an example interface that facilitates dead band normalization in accordance with example embodiments. As explained below, in a syringe-type fluid delivery mechanism, a drive assembly can be selectively engaged and disengaged from the plunger to allow for operating the pump without fluid movement to obtain unloaded measured data for dead band normalization in accordance with example embodiments.

FIGS. 12-15B illustrate another example fluid delivery device having a different example interface from FIGS. 4-11 to facilitate dead band normalization in accordance with example embodiments. As explained below in connection with FIGS. 12-15B, in a rotational metering-type fluid delivery mechanism, a gearbox and output gear coupling to a pump drive mechanism allows for the pump to be driven without moving fluid to obtain data in a dead band region 100 of an aspirate or dispense operation to obtain unloaded measured data for dead band normalization in accordance with example embodiments.

FIG. 4 is a side view of an example wearable fluid delivery device 10 constructed to perform improved occlusion detection in accordance with an example embodiment. The fluid delivery device 150 comprises a baseplate 152, a cover 154, and an insertion mechanism 156 in an undeployed position.

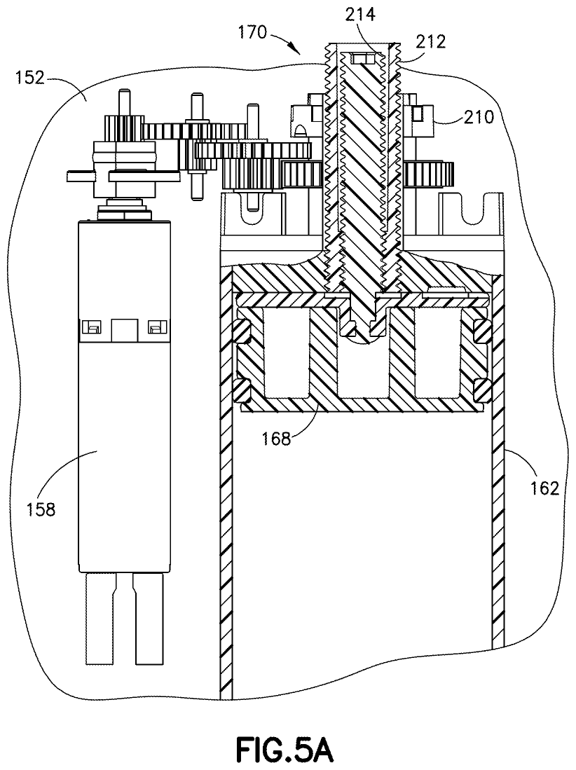

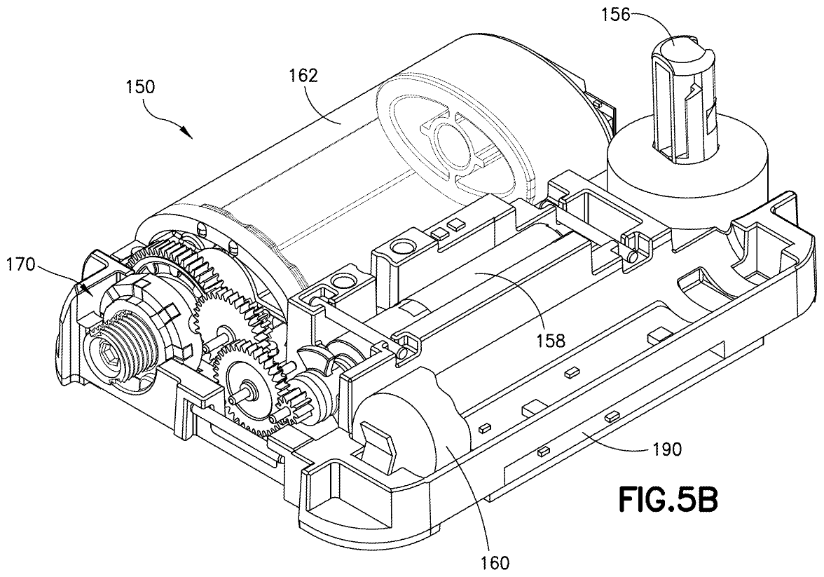

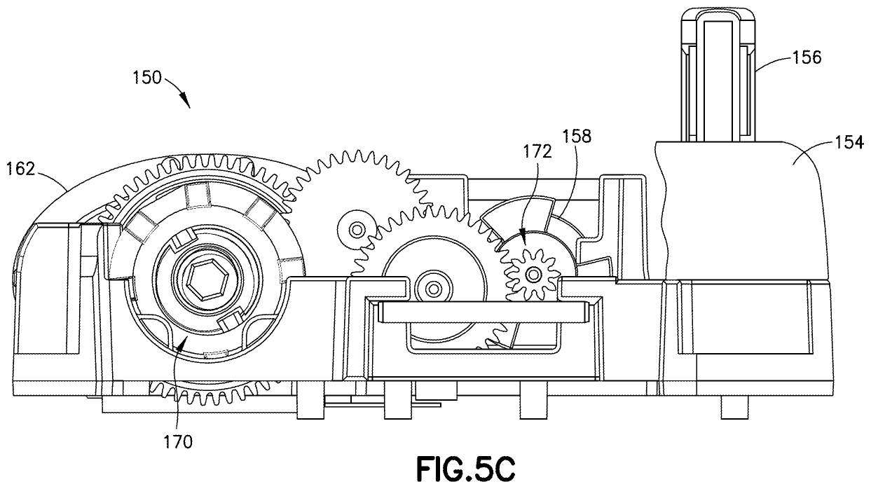

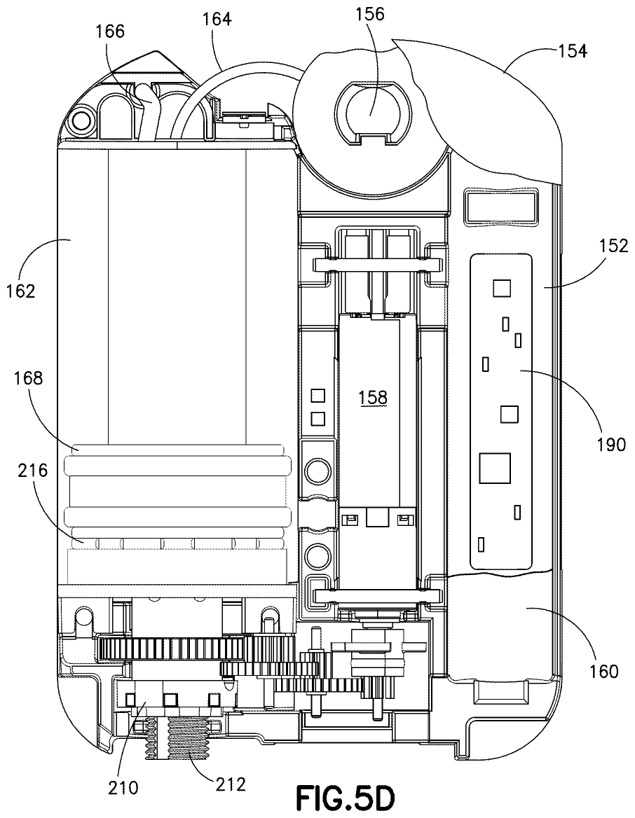

FIGS. 5A, 5B, 5C and 5D are, respectively, a partial top view, a perspective view, a side view, and a top view of the fluid delivery device 150 of FIG. 4 with the cover 154 removed. The baseplate 152 supports the insertion mechanism 156, a motor 158, a power source such as a battery 160, a control board 190, and a reservoir 162 or container for storing a fluid to be delivered to a user via an outlet fluid path 164 from and outlet port of reservoir to the insertion mechanism 156. The reservoir 162 can also have an inlet port connected via an inlet fluid path 166 to a fill port (e.g., provided in the baseplate 12). The reservoir 162 contains a plunger 168 having a stopper assembly. The proximal end of the reservoir 162 is also provided with a plunger driver assembly 170. The plunger driver assembly 170 can be a telescoping, simultaneously counter-rotating sleeve screw 212 and center screw 214, a gear anchor 174, a nut 210 that is rotated via a gear train 172 connected to the motor 158 and gearbox 184. It is to be understood that the plunger driver assembly 170 can comprise different components for pushing and extracting the plunger 168 within the reservoir 162.

FIG. 6 is a block diagram of example components of a fluid delivery device. The cover/housing or device 150 housing is indicated at 154. The device 150 has skin retention subsystem 180 such as an adhesive pad to connect the device 10 to a user's skin. The fluid delivery device 10 further comprises the reservoir 162, the insertion mechanism 156, and a fluid displacement module 182 that can include the motor 158, motor housing and gearbox 184, gear train 178, pump mechanism (e.g., plunger driver assembly 170), and outlet path 164. The fluid delivery device further comprises electrical components such as a power module (e.g., battery 160), and an electrical module 190 comprising a controller 192, a motor driver 194, optional sensing module 196 to sense fluid flow conditions (e.g. occlusion), optional audio driver 198 (e.g., to indicate dosing in progress, low reservoir, occlusion, successful pairing with external device, or other condition), and an optional visual driver 200, and an optional wireless driver 202 for wireless communication between the fluid delivery device and an optional remote pump control device 203 (e.g., a smartphone or dedicated controller). As described below, the controller 192 can be programmed or otherwise configured to perform the improved occlusion detection of the example embodiments of the technical solution described herein.

FIGS. 7A, 7B, 7C and 7D are perspective top views of a fluid delivery device with the cover removed and showing different stages of filling the reservoir 162. A fluid filled chamber 204 in the reservoir 162 is defined by a distal or front side of the plunger 168 and that plunger is configured to seal the fluid from entering the portion of reservoir defined by proximal or rear side of plunger so that there is no contact with the plunger driver assembly 170 or gear anchor 174 with the fluid being delivered from the reservoir.

In FIG. 7A, the reservoir 162 is empty of any fluid and the plunger 168 is at its most distal position. The plunger driver assembly 170 is shown fully retracted in FIGS. 7A through 7D. A user can insert the needle of a filled syringe 176 into a fill port (not shown) provided in the baseplate 152 that has an inlet fluid path 166 from the fill port to the reservoir 162 as shown in FIG. 5D. As fluid is transferred from the syringe 176 to the reservoir 162 via the inlet fluid path 166, the volume of a fluid chamber defined in the reservoir 162 by the front surface of the plunger 168 increases, as shown in FIGS. 7B, 7C and 7D respectively. The plunger 168 has a stopper assembly 169 to prevent leakage of any fluid retained in a fluid chamber portion 204 of the reservoir 162. The stopper assembly 169 can comprise, for example, an elastic material similar to a syringe stopper.

As shown in FIGS. 2A-7B, a gear anchor 174 has an aperture 222 to receive a first portion 210a of a nut 210. The aperture 222 has threads 224 that are configured to cooperate with the outer threads 212a of the sleeve screw 212. The number of threads 84 can be adjusted to balance torque and movement stability. The number of threads 224 can be added without negatively affecting length (i.e., only a small change in the drive nut geometry is needed). A recessed rear surface 226 is configured to rotatably receive the first portion 210a of the nut 210. The gear anchor 174 has front surface 228 that can abut a plunger pusher 216 when the plunger driver assembly 170 fully retracted and the reservoir is filled (e.g., as shown in FIG. 7D) but is not required to do so depending on the dimensions of the reservoir 162 and the plunger driver assembly 170.

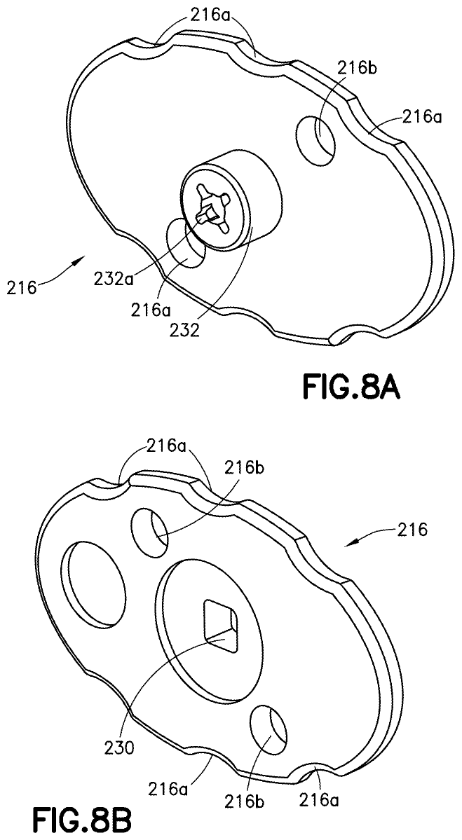

FIGS. 8A and 8B are, respectively, a front perspective view and a rear perspective view of the plunger pusher 216. The plunger pusher 216 has a detent 230 on a rear surface thereof to receive a keying feature 214b on the center screw 214. An optional protrusion 232 the front surface of the plunger pusher 216 impacts the rear surface of plunger 168. The pusher 216, together with or alternatively the cap or plunger driver assembly 170 on the reservoir 22, is provided with feature(s) to allow air venting. For example, an air venting feature can be provided along at least a portion of the perimeter of the pusher 216 and be in the form of a scalloped edge comprising notches 216a. When notches 216a are provided on the perimeter of the pusher 216, these features can be arranged to minimize axial translation friction by biasing design and tolerances for edges around a few of these features 216a to be more proud of the remaining notch edges so as to make first contact with the internal reservoir barrel face to prevent rotation. The pusher 216 can also be provided with one or more through holes 216b in a plate-like portion of the pusher for venting.

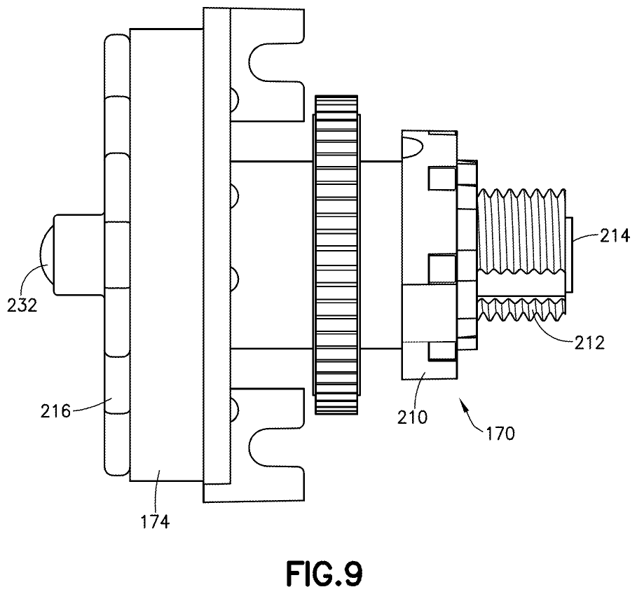

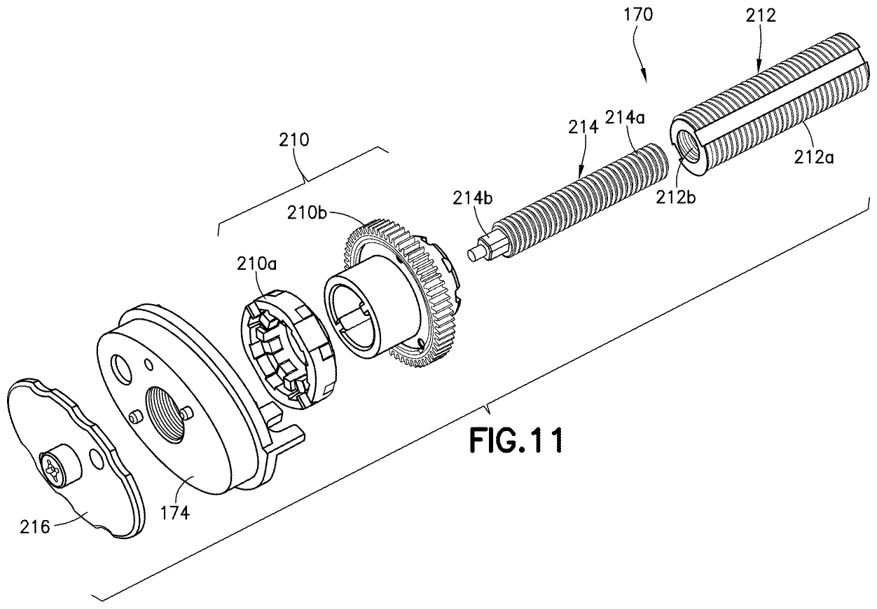

FIG. 9 is a side view of the plunger driver assembly 30 in a retracted position relative to the gear anchor 34. With reference to FIGS. 9 and 11, the plunger driver assembly 170 comprises the nut 210 having teeth on a portion thereof that engages the gear train 172 and motor 158. A first portion of the nut 210a is rotationally received in gear anchor 174. Inner threads in the nut 210 engage outer threads 212a of the sleeve screw 212. Inner threads 212b in the cavity of the sleeve screw 212 engage outer threads 214a of the center screw 214. As described with respect to FIG. 11, the distal end of the center screw 214 is provided with a keying feature 214b that engages a detent 230 on the plunger pusher 216.

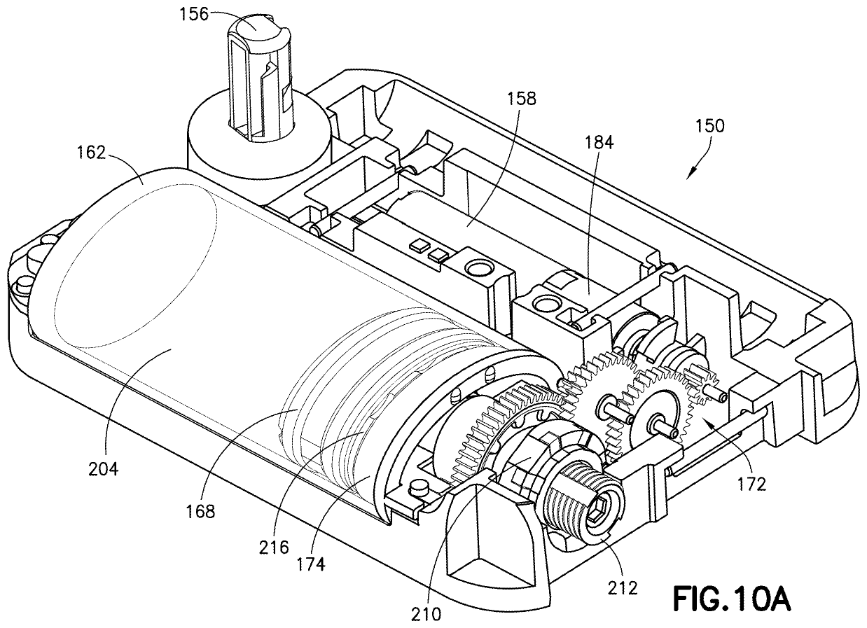

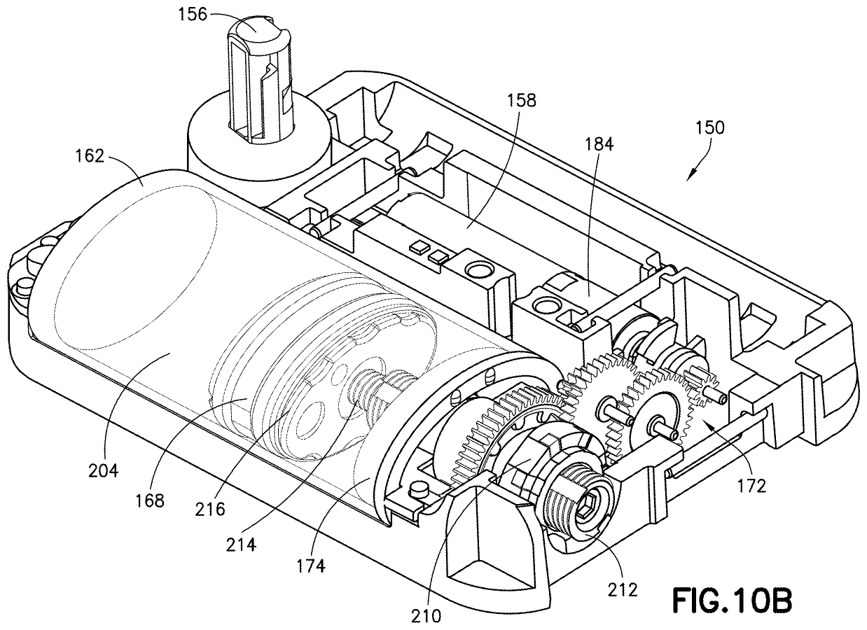

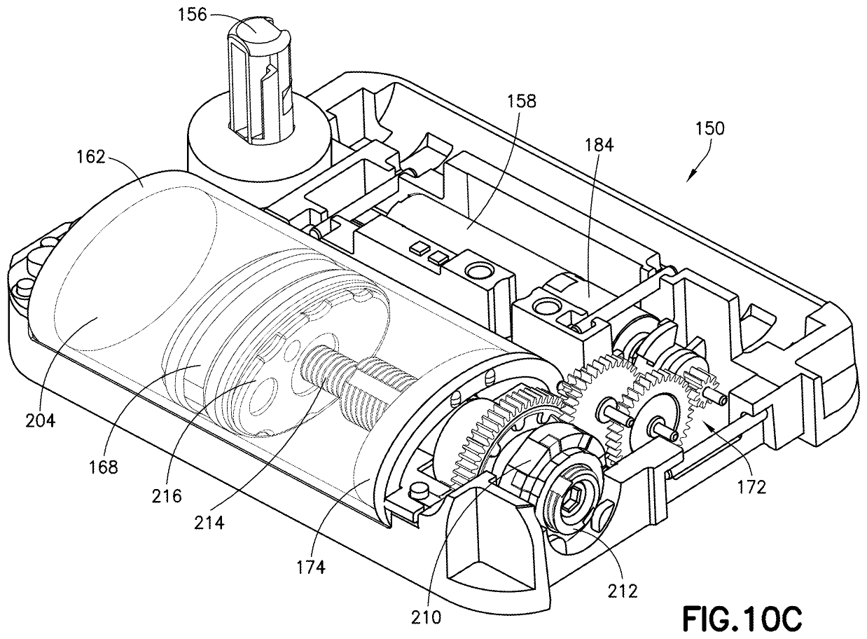

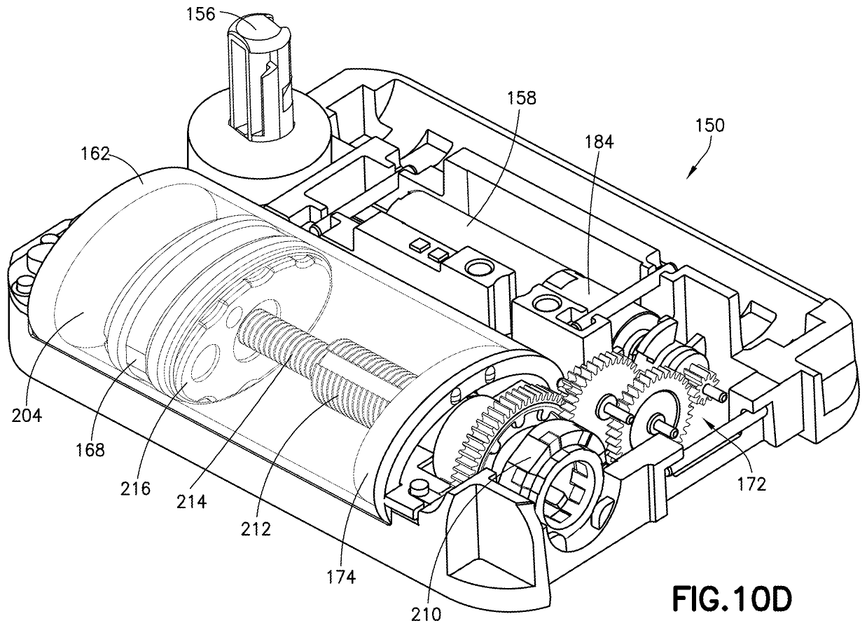

FIGS. 10A, 10B, 10C and 10D are perspective top views of a fluid delivery device with the cover removed and showing different stages of discharging fluid from a reservoir via a plunger drive assembly 170. In FIG. 10A, the plunger drive assembly 170 is in a fully retracted position and the volume of the fluid filled chamber portion 204 of the reservoir 162 is maximized. In FIGS. 10B, 10C and 10D, the nut 210 is being rotated by the motor and gearbox 158 and the intermediate power transmission gear train 172 via engagement of its teeth 210b. The inner threads 210b of the nut and the aperture threads 224 of the gear anchor 174 cooperate with the outer threads 212a of the sleeve screw 212 to advance the sleeve screw 212 through the nut 210 and the gear anchor 174 and into the reservoir 162. Simultaneously, the rotation of the sleeve screw 212 causes non-rotational advancement of the center screw 214 which is keyed to the plunger pusher 216. As a result, the plunger 168 is advanced distally as plunger pusher 216 is advanced distally to abut the plunger 168.

With reference to FIGS. 8B and 11, the keying feature 214b on the center screw 214 and its corresponding detent 230 on the rear surface of the plunger pusher 216 provides an anti-rotation mechanism for the plunger pusher 216 relative to the reservoir 162 when the nut of the plunger drive assembly 170 is being rotated by the motor and gearbox 158 and the intermediate power transmission gear train 172. The center screw 214 can be provided with a keyed feature to engage the plunger pusher 216. This keyed feature can either engage with a non-circular plunger pusher geometry, whereby rotation is prevented by geometry, or can be engaged with an intermediate structure that acts to prevent rotation in the operating syringe barrel-type reservoir 22. For example, the distal end of the center screw 214 can be dimensioned and/or shaped to engage a corresponding dimensioned and/or shaped detent or indent 230 in the plunger pusher 216 that prevents any limited rotation imparted on the center screw 214 by the other components 210 and 212 from causing rotation of the plunger pusher 216 relative to the inner walls of the reservoir 162.

As stated above, an optional protrusion 232 on the front surface of the plunger pusher 216 impacts the rear surface of plunger 168. In accordance with an aspect of the technical solution, the front surface of the plunger pusher 216 can be controllably engaged with or abut the rear surface of plunger 168 when the plunger drive assembly 170 is driven by the motor and gearbox 158 to advance toward the distal end of the fluid chamber portion 204 (e.g., to dispense fluid from the chamber), and disengaged or distanced from the rear surface of plunger 168 when the plunger drive assembly 170 is driven by the motor and gearbox 158 to retract toward the gear anchor 174, to provide an interface to facilitate dead band normalization (e.g., to obtain unloaded measured data to which loaded measured data can be normalized) in accordance with illustrative embodiments of the technical solution. A controlled minor retraction of the plunger pusher 216 from the plunger 168 during a dispense operation, for example, allows for dead band normalization to be determined by the controller 192 for comparison with and more accurate analysis of a measured pump parameter obtained during a subsequent dispense to remove noise and more accurately determine a pump motor condition such as catheter occlusion, air in fluid path or motor malfunction, among other fluid delivery device conditions that impact fluid path pressure characteristics. Also, the controller can be configured to generate the unloaded measured data prior to gathering of any loaded measured data by incrementing through a known number of dispense cycles in which the pusher has not yet hit the plunger.

The example embodiments of dead band normalization algorithm are also useful with respect to positive displacement pumps. A positive displacement pump is understood to be a type of pump that works on the principle of filling a chamber (e.g., with liquid medication from a reservoir) in one stage and then emptying the fluid from the chamber (e.g., to a delivery device such as a cannula deployed in a patient) in another stage. For example, a reciprocating plunger-type pump or a rotational metering-type pump can be used. In either case, a piston or plunger is retracted from a chamber to aspirate or draw medication into the chamber and allow the chamber to fill with a volume of medication (e.g., from a reservoir or cartridge of medication into an inlet port). The piston or plunger is then re-inserted into the chamber to dispense or discharge a volume of the medication from the chamber (e.g., via an outlet port) to a fluid pathway extending between the pump and a cannula in the patient.

For illustrative purposes, reference is made to an example rotational metering-type pump described in commonly owned WO 2015/157174, the content of which is incorporated herein by reference in its entirety. The illustrative system diagram in FIG. 15A can also illustrate example components in the pump of FIGS. 4-11 as well as other types of pumps.

With reference to FIGS. 12, 13, 14A, 14B, 14C, 14D, 15A and 15B, an example rotational metering-type infusion pump (e.g., a wearable fluid delivery device such as an insulin patch pump) comprises a pump assembly 20 which can be connected to a DC motor and gearbox assembly 33 (FIG. 14D) to rotate a sleeve 24 in a pump manifold 22 (FIG. 14D). A helical groove 26 is provided on the sleeve. A coupling pin 28 connected to a piston 30 translates along the helical groove to guide the retraction and insertion of the piston 30 within the sleeve 24, respectively, as the sleeve 24 rotates in one direction and then rotates in the opposite direction. The sleeve has an end plug 34. Two seals 32, 36 on the respective ends of the piston and end plug that are interior to the sleeve 24 define a cavity or chamber 38 when the piston 30 is retracted, as depicted in FIG. 3A, following an aspirate stroke and therefore ready to dispense. The volume of the chamber 38 therefore changes depending on the degree of retraction of the piston 30. The volume of the chamber 38 is negligible or essentially zero when the piston 30 is fully inserted and the seals 32, 36 are substantially in contact with each other following a dispense stroke, as depicted in FIG. 3B, and therefore ready to aspirate. Two ports 44, 46 are provided relative to the pump manifold 22, including an inlet port 44 through which medication can flow from a reservoir 70 (FIG. 4A) for the pump 64 (FIG. 4A), and an outlet port 46 through which the medication that has been drawn into the chamber 38 (e.g., by retraction of the piston 30 during an aspirate stage of operation) can be dispensed from the chamber 38 to, for example, a fluid path to a cannula 72 (FIG. 4A) in the patient by re-insertion of the piston 30 into the chamber 38.

With continued reference to FIGS. 12, 13 and 14A-14C, the sleeve 24 can be provided with an aperture (not shown) that aligns with the outlet port 46 or the inlet port 44 (i.e., depending on the degree of rotation of the sleeve 24 and therefore the degree of translation of the piston 30) to permit the medication in the chamber 38 to flow through the corresponding one of the ports 44, 46. A pump measurement device 78 (FIG. 15A) such as a sleeve rotational limit switch can be provided which has, for example, an interlock 42 and one or more detents 40 on the sleeve 24 or its end plug 34 that cooperate with the interlock 42. The interlock 42 can be mounted to the manifold 22 at each end thereof. The detent 40 at the end face of sleeve 24 is adjacent to a bump 48 of the interlock 42 when the pump 64 is in a first position whereby a side hole in the sleeve 24 is aligned with the inlet port 44 to receive fluid from the reservoir 70 into the chamber 38. Under certain conditions, such as back pressure, it is possible that friction between the piston 30 and the sleeve 24 is sufficient to cause the sleeve 24 to rotate before the piston 30 and coupling pin 28 reach either end of the helical groove 26. This could result in an incomplete volume of liquid being pumped per stroke. In order to prevent this situation, the interlock 42 prevents the sleeve 24 from rotating until the torque passes a predetermined threshold, as shown in FIG. 14A. This ensures that piston 30 fully rotates within the sleeve until the coupling pin reaches the end of the helical groove 26. Once the coupling pin 28 hits the end of the helical groove 26, further movement by the DC motor and gearbox assembly or other type of pump and valve actuator 66 (Fig. increases torque on the sleeve 24 beyond the threshold, causing the interlock 42 to flex and permit the detent 40 to pass by the bump 48. At the completion of rotation of the sleeve 24 such that its side hole is oriented with the cannula 72 or outlet port 46, the detent 40 moves past the bump 48 in the interlock 42, as shown in FIG. 14B. Another sleeve feature 41 can be provided to engage an electrical switch (e.g., an end-stop switch 90 provided on a printed circuit board 92 and disposed relative to the sleeve and/or end plug 34 to cooperate with the pump measurement device 78 as shown in FIG. 14C).

A gap between the piston 30 and the output gear 39 of the gearbox (e.g., between a tab 31 at the end of the piston and a sl5t 35 in the output gear 39) provides a beneficial interface for dead band normalization since it is close to a fluid driving interface that is capable at least temporarily of moving yet without moving the fluid during a fluid movement operation. For example, as illustrated in FIG. 1D, even when a drive mechanism operates a pump piston 30 in a pump aspirate or dispense operation that moves fluid, an initial point 100 after motor startup in a new direction is similar to unloaded measured data, and loaded measured data can be normalized to this unloaded measured data.

FIG. 14D illustrates part of a manifold 22 having a motor and gearbox 33 that cooperates with the pump assembly 20. The motor and gearbox 33 includes an opening 43 that can receive a rotational limit switch. In this manner, output gear 39, which is internal to the gearbox housing, can access and engage the flexures of a limit switch. Motor and gearbox 33 also include an axial retention snap 37 so that the pump assembly 20 may be snap-fit to the motor and gearbox 33. Motor and gearbox 3 includes a rotational key 41 within a pump-receiving socket 43 to receive pump assembly 20 and prevent rotation of the pump assembly relative to the motor and gearbox 33. Output gear 39 includes a slot 35 adapted to receive a tab 31 provided on the piston 30. When assembled, tab 31 is received into slot 35 so that the output gear 39 can transmit torque to the piston 30. As the output gear 39 rotates, the pump piston tab 31 both rotates and slides axially in the slot 35 to provide a useful interface with which to obtain unloaded measured data for dead band normalization with loaded measured data corresponding to when the piston is moving fluid to or from the chamber 38. Metal spring flexures on the motor connections and limit switches are used to make electrical contact with pads on the circuit board 92 during final assembly.

Alternatively, an interface that can facilitate dead normalization in the example rotational metering-type infusion pump can be designed with respect to the helical groove 26 and coupling pin 28. During a discharge stroke, the piston 30 is turned in a first rotational direction and is driven along the helical path of the helical groove 26 in the sleeve 24 via the coupling pin 28. The pump piston 30 translates away from the gearbox while rotating, expelling fluid from the pump chamber 38 and out of the cannula port 1356. During the discharge stroke, friction between the port seals and the outside diameter of the sleeve 24 is sufficient to ensure that the sleeve does not rotate during this portion of the pump cycle. During a valve state change after the discharge stroke, the coupling pin 28 reaches the distal end of helical groove 26 and torque continues to be transmitted from the output gear, to the pump piston 30, and to the sleeve 24 via the coupling pin 28. The sleeve 24 and pump piston 30 rotate as a unit with no relative axial motion. The side hole on the sleeve 24 moves from the outlet port 46 to the inlet port 44. During an intake stroke, the output gear turns the pump piston 30 and the piston is translated axially relative to the sleeve 24 due to interaction of the coupling pin 28 within the helical groove 26. The pump piston 30 translates toward the gearbox, pulling fluid from the reservoir into the pump chamber via the inlet port 44. During a valve state change after the intake stroke, the coupling pin 28 reaches the upper end of helical groove 26, the pump motor continues to deliver torque, causing the sleeve 24 and piston 30 to rotate together as a unit with no relative axial motion and the side hole of the sleeve 24 to move from alignment with the inlet port 44 to alignment with the outlet port 46. The helical groove 26 and coupling pin 28 can be configured by extending the groove or otherwise altering dimensions or slope of the groove to provide a dead region (e.g., 100 in FIG. 2) within the fluid movement operation wherein drive mechanism component(s) operate but do not move fluid to provide an interface for dead normalization.

In accordance with another example embodiment, a fluid delivery device can have a drive mechanism employing one or more cams that can provide a beneficial interface for dead band normalization in accordance with the present technical solution. Unloaded measured data for dead band normalization can be obtained, for example, using a dead region provided by a cam and cam follower. At some point during a fluid movement operation wherein an actuator with cam follower is being controlled to rotate relative to a cam, the cam follower's advancement along a flat surface of the cam does not result in a related gear or other component connected to the cam being operated to move fluid during that fluid movement operation.

Regardless of the type of actuator and drive mechanism employed in a fluid delivery device such as wearable medication infusion pump, dead band normalization advantageously uses an unloaded region or portion in a positive displacement pump fluid movement operation to obtain unloaded measured data related to fluid movement (e.g., pressure, flow rate, and so on) with which to normalize loaded measured data related to that fluid movement operation. The resulting normalized measured data is advantageous because signal noise related to the actuator and impact of external noise factors (e.g., environmental factors and part-to-part variation) is removed, allowing for more accurate occlusion detection using the normalized measured data. Another benefit of dead band normalization in accordance with the technical solution and example embodiments described herein is that the unloaded and loaded measured data signals used for dead band normalization are processed very locally, that is, close to a particular fluid movement event (e.g., a particular aspirate stroke or a dispense stroke). It is to be understood that this local or proximal operation is not limited by any particular timing or order of operation for obtaining the loaded and unloaded measured data during a particular fluid movement event or operation.

It will be understood by one skilled in the art that this disclosure is not limited in its application to the details of construction and the arrangement of components set forth in the above description or illustrated in the drawings. The embodiments herein are capable of other embodiments, and capable of being practiced or carried out in various ways. Also, it will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings. Further, terms such as up, down, bottom, and top are relative, and are employed to aid illustration, but are not limiting.

The components of the illustrative devices, systems and methods employed in accordance with the illustrated embodiments can be implemented, at least in part, in digital electronic circuitry, analog electronic circuitry, or in computer hardware, firmware, software, or in combinations of them. These components can be implemented, for example, as a computer program product such as a computer program, program code or computer instructions tangibly embodied in an information carrier, or in a machine-readable storage device, for execution by, or to control the operation of, data processing apparatus such as a programmable processor, a computer, or multiple computers.

A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network. Also, functional programs, codes, and code segments for accomplishing the illustrative embodiments can be easily construed as within the scope of claims exemplified by the illustrative embodiments by programmers skilled in the art to which the illustrative embodiments pertain. Method steps associated with the illustrative embodiments can be performed by one or more programmable processors executing a computer program, code or instructions to perform functions (e.g., by operating on input data and/or generating an output). Method steps can also be performed by, and apparatus of the illustrative embodiments can be implemented as, special purpose logic circuitry, e.g., an FPGA (field programmable gate array) or an ASIC (application-specific integrated circuit), for example.

The various illustrative logical blocks, modules, and circuits described in connection with the embodiments disclosed herein may be implemented or performed with a general purpose processor, a digital signal processor (DSP), an ASIC, a FPGA or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example, semiconductor memory devices, e.g., electrically programmable read-only memory or ROM (EPROM), electrically erasable programmable ROM (EEPROM), flash memory devices, and data storage disks (e.g., magnetic disks, internal hard disks, or removable disks, magneto-optical disks, and CD-ROM and DVD-ROM disks). The processor and the memory can be supplemented by, or incorporated in special purpose logic circuitry.

Those of skill in the art would understand that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof

Those of skill would further appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of claims exemplified by the illustrative embodiments. A software module may reside in random access memory (RAM), flash memory, ROM, EPROM, EEPROM, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. In other words, the processor and the storage medium may reside in an integrated circuit or be implemented as discrete components.

Computer-readable non-transitory media includes all types of computer readable media, including magnetic storage media, optical storage media, flash media and solid state storage media. It should be understood that software can be installed in and sold with a central processing unit (CPU) device. Alternatively, the software can be obtained and loaded into the CPU device, including obtaining the software through physical medium or distribution system, including, for example, from a server owned by the software creator or from a server not owned but used by the software creator. The software can be stored on a server for distribution over the Internet, for example.

The above-presented description and figures are intended by way of example only and are not intended to limit the illustrative embodiments in any way except as set forth in the following claims. It is particularly noted that persons skilled in the art can readily combine the various technical aspects of the various elements of the various illustrative embodiments that have been described above in numerous other ways, all of which are considered to be within the scope of the claims.

1. A fluid delivery device comprising:
a pump comprising a chamber of fluid, and a drive mechanism configured to control movement of a designated volume of fluid with respect to the chamber during a fluid movement operation; and
a processing device configured, during a fluid movement operation, to generate measured data comprising unloaded measured data obtained during a portion of the fluid movement operation wherein the pump does not move fluid, and loaded measured data obtained while the pump is moving fluid during the fluid movement operation, the measured data being indicative of fluid movement in the pump, and to normalize the loaded measured data to the unloaded measured data.

2. The fluid delivery device of claim 1, wherein the processing device is further configured to analyze the normalized loaded measured data to determine if it satisfies a designated metric related to pressure in the infusion device that indicates occlusion.

3. The fluid delivery device of claim 1, wherein the processing device is further configured, during a subsequent fluid movement operation by the pump to
generate unloaded measured data during a portion of the subsequent fluid movement operation wherein the pump does not move fluid,
generate loaded measured data while the pump is moving fluid during the subsequent fluid movement operation, the measured data being indicative of fluid movement in the pump, and
normalize the loaded measured data to the unloaded measured data.

4. The fluid delivery device of claim 1, wherein the fluid movement operation is an incremental operation among a plurality of fluid movement operations to dispense fluid from the chamber or aspirate fluid into the chamber.

5. The fluid delivery device of claim 1, wherein the processing device is further configured to normalize the loaded measured data to the unloaded measured data for each fluid movement operation of the fluid delivery device, or least for a selected subset of fluid movement operations of the fluid delivery device.

6. The fluid delivery device of claim 1, wherein the fluid delivery operation is chosen from an aspirate operation to draw fluid into the chamber and a dispense operation to expel fluid from the chamber.

7. The fluid delivery device of claim 1, wherein the measured data indicates a fluid characteristic chosen from fluid pressure and fluid flow rate.

8. The fluid delivery device of claim 1, wherein the pump is a syringe-type pump having a barrel as the chamber and a plunger and the drive mechanism is operable to selectively drive the plunger to dispense fluid from the barrel, and the processing device is configured to generate the unloaded measured data before the measured data indicates that fluid pressure or flow rate has begun to increase from driving the plunger by the drive mechanism during the fluid movement operation.

9. The fluid delivery device of claim 1, wherein the pump is characterized by an interface comprising at least one or more components in the drive mechanism and the operation of which causes the portion within a fluid movement operation wherein the pump does not move fluid to occur.

10. The fluid delivery device of claim 9, wherein the pump is a syringe-type pump having a barrel as the chamber and the interface comprises a plunger, the drive mechanism being operable to selectively drive the plunger to dispense fluid from the barrel, and the processing device is configured to generate the unloaded measured data during a dispensing fluid movement operation by temporarily retracting the plunger in the barrel a nominal amount.

11. The fluid delivery device of claim 9, wherein the pump is a syringe-type pump having a barrel as the chamber and the interface comprises a plunger, the drive mechanism being operable to selectively drive the plunger to dispense fluid from the barrel, and the processing device is configured to generate the unloaded measured data prior to gathering of loaded measured data by incrementing through a known number of dispense cycles in which the pusher has not yet hit the plunger.

12. The fluid delivery device of claim 9, wherein the pump is a syringe-type pump having a barrel as the chamber and the interface comprises a plunger, the drive mechanism being operable to selectively drive the plunger to dispense fluid from the barrel, and the processing device is configured to generate the unloaded measured data during an aspirating fluid movement operation by manual or externally controlled filling of the barrel via an inlet port to the barrel, and to generate the loaded measured data during the aspirating fluid movement operation by controlling the pump to temporarily retract the plunger within the barrel.

13. The fluid delivery device of claim 9, wherein the pump is a syringe-type pump having a barrel as the chamber and a plunger, the interface comprises a pusher coupled to the drive mechanism, the drive mechanism being operable to selectively drive the pusher to abut the plunger to dispense fluid from the barrel, and the processing device is configured to generate the unloaded measured data during a dispensing fluid movement operation by temporarily retracting the pusher in the barrel.

14. The fluid delivery device of claim 9, wherein the pump is a rotational metering-type pump comprising an inlet port and an outlet port and wherein the drive mechanism is connected to a pump motor via a gearbox and the chamber has at least one aperture, the drive mechanism being operable to selectively drive a piston to dispense fluid from or aspirate fluid into the chamber and to control cooperation of the at least one aperture with the inlet port during an aspirating fluid movement operation and with the outlet port during a dispensing fluid movement operation, the interface comprising a feature on the drive mechanism that is configured to cooperate with the gearbox to enable the drive mechanism to not move fluid with respect to the chamber during at least a portion of the aspirating fluid movement operation and the dispensing fluid movement operation.

15. The fluid delivery device of claim 9, wherein the pump is a rotational metering-type pump and the interface comprises a pin on a piston that is controllably inserted and retracted within a sleeve and a helical groove in the sleeve, the drive mechanism being operable to rotate the sleeve causing the for controlling fluid volume in the chamber via a helical groove in the sleeve to guide the pin to translate along the helical groove to guide the retraction and insertion of the piston within the sleeve to control fluid volume of the chamber, the pin and/or groove being configured to enable the piston to not move fluid with respect to the chamber during at least a portion of a fluid movement operation.

16. The fluid delivery device of claim 9, wherein the interface comprises a cam coupled to the drive mechanism, and the processing device is configured to generate the unloaded measured data during a fluid movement operation when a cam follower connected to an actuator for the drive mechanism traverses at least part of a flat portion of the cam resulting in no fluid movement during the fluid movement operation.

17. The fluid delivery device of claim 1, wherein the pump has a reservoir as the chamber, a plunger and a drive mechanism operable to selectively drive the plunger to dispense fluid from the reservoir, and the processing device is configured with baseline data related to a designated waveform of the measured data during fluid movement operations, the waveform having a dead portion therein corresponding to when fluid pressure or rate from driving the plunger by the drive mechanism has not yet begun to increase, the processing device being configured to analyze the measured data using the baseline data to determine when to generate the unloaded measured data during a fluid dispense operation.
